SEQUENCE LISTING (11 sequences)

SEQ ID NO: 1 - protein, 288 aa, Artificial Sequence
  Synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 2 - protein, 208 aa, Artificial Sequence [sequence omitted]

SEQ ID NO: 3 - protein, 126 aa, Artificial Sequence
  Synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGAQIYVIDPEPCPDSD

SEQ ID NO: 4 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 5 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 7 - protein, 607 aa, Artificial Sequence
  Synthetic construct
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPIN

SEQ ID NO: 8 - protein, 214 aa, Artificial Sequence
  Synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 55 aa, Artificial Sequence
  Synthetic construct
GTSHLVKCAEKEKTFCVNGGECFMVKDLSNPSRYLCKCPNEFTGDRCQNYVMASF

SEQ ID NO: 10 - protein, 9 aa, Artificial Sequence
  Synthetic construct
TYLPANASL

SEQ ID NO: 11 - protein, 76 aa, Homo sapiens
ITKAGLQVYNKCWKFEHCNFNDVTTRLRENELTYYCCKKDLCNFNEQLENGGTSLSEKTVLLLVTPFLAAAWSLHP

(19) United States
(12) Patent Application Publication
(10) Pub. No.: US 2023/0181633 A1
(43) Pub. Date: Jun. 15, 2023

(54) METHODS OF TREATING CANCER USING A COMBINATION OF TUMOR MEMBRANE VESICLES AND METFORMIN

(71) Applicants: METACLIPSE THERAPEUTICS CORPORATION, Atlanta, GA (US); EMORY UNIVERSITY, Atlanta, GA (US)

(72) Inventors: Periasamy SELVARAJ, Lilburn, GA (US); Luis Enrique MUNOZ, Decatur, GA (US); Ramireddy BOMMIREDDY, Duluth, GA (US); Christopher D. PACK, Atlanta, GA (US); Sampath RAMACHANDIRAN, Duluth, GA (US)

(21) Appl. No.: 17/427,380

(22) PCT Filed: Jan. 31, 2020
(86) PCT No.: PCT/US2020/016106

§ 371 Date: Jul. 30, 2021

Related U.S. Application Data
(60) Provisional application No. 62/799,381, filed on Jan. 31, 2019.

(51) Int. Cl. A61K 35/13; A61K 31/155; C07K 14/71; C07K 16/28; A61P 35/00

(52) U.S. Cl. CPC A61K 35/13; A61K 31/155; A61P 35/00; C07K 14/71; C07K 16/2818; C07K 16/2896

(57) ABSTRACT

Disclosed herein is a method for treating a subject having, or at risk of having, a cancer, comprising administering to the subject a therapeutically effective amount of a tumor membrane vesicle (TMV) and a metformin. In some embodiments, the TMV comprises a B7-1 and/or IL-12 molecule anchored to a lipid membrane (e.g., by a GPI anchor). In some embodiments, the methods can further comprise administering an immune checkpoint inhibitor. The methods are useful for reducing tumor size and metastasis, and in improving anti-tumor immune responses.

### CROSS REFERENCE TO RELATED APPLICATIONS

This is a national stage application filed under 35 U.S.C. §371 of PCT/US2020/016106, filed on Jan. 31, 2020, which claims the benefit of U.S. Provisional Application No. 62/799,381, filed on Jan. 31, 2019, each of which are incorporated herein by reference in their entirety.

### STATEMENT REGARDING FEDERALLY SPONSORED RESEARCH OR DEVELOPMENT

This invention was made with government support under CA202763 awarded by the National Institutes of Health. The U.S. Government has certain rights in this invention.

### INCORPORATION-BY-REFERENCE OF MATERIAL SUBMITTED ELECTRONICALLY

Incorporated by reference in its entirety herein is a computer readable sequence listing submitted concurrently herewith and identified as follows: One 22 kilobytes ASCII (Text) file named “10798-004US1_2022_03_21_Sequence,” created on Mar. 21, 2022.

### BACKGROUND

According to the American Cancer Society (ACS), nearly 14 million Americans have a history of cancer, with breast cancer having some of the highest incidence and mortality rates. See Howlader et al., A. SEER Cancer Statistics Review, 1975-2013, National Cancer Institute. Triple negative breast cancer (TNBC) afflicts up to 50,000 women per year in the US, typically at a younger age than other breast cancers and with a poorer overall prognosis. This poor clinical outcome is attributed to a lack of a defined target, high patient-to-patient heterogeneity, and an aggressive phenotype. Even with conventional radiation and chemotherapy regimens, patients have poor prognosis, experiencing early, frequent relapses in comparison to other breast cancers. In addition, a high level of intratumoral as well as patient-to-patient heterogeneity is observed among triple negative patients, making it even more difficult to treat. See Gerlinger et. al., The New England Journal of Medicine, 366:883-92 (2012). Therapies effective for other cancers, even other breast cancers, frequently prove ineffective at treating TNBC. Thus, it is difficult to know whether a known anti-cancer therapy will be therapeutic in TNBC patients. TNBC is a clear area of significant unmet medical need, and new therapies that address patient-to-patient variation in tumor targets are critically required.

Immune dysfunction is associated with tumor progression and metastasis in cancer patients. Tumors evade the host immune system by numerous mechanisms such as suppression, anergy or deletion of effector T cells.

Recent developments in cancer therapies include the use of tumor membrane vesicles (TMVs) prepared from a patient’s own tumor (see US2015/0071987, U.S.). The tumor membrane vesicles can be further modified by incorporating immunostimulatory agents (ISMs). Use of autologous tumor tissue from the patient incorporates the patient’s unique immune signature into the vaccine design and overcomes the issue of heterogeneity within a single tumor and patient-to-patient variation in gene mutations.

Interestingly, a recent report from the American Diabetes Association (ADA) concluded that cancer and diabetes are diagnosed in the same patient more frequently than what would be expected by chance. See Giovannucci, et al., Diabetes Care, 33:7 (2010). The most commonly prescribed drug for type 2 diabetics is metformin. Nathan, et al., Diabetes Care, 32:1 (2008). Metformin is a biguanide originally derived from the French lilac (Galega officinalis). While the complete mechanism of action behind metformin is not well understood, it is known that it can reduce levels of both circulating glucose and insulin mainly by reducing hepatic glucose output. Shaw, et al., Science, 310:5754 (2005). A growing body of evidence has suggested that metformin can induce anti-cancer responses by influencing both immune responses and tumor cell growth. Metformin can exert antineoplastic activity by activating AMPK, and inhibiting mTOR, Ras, and PI3K signaling pathways. Nair, et al., J. Biol. Chem., 289:40 (2014); Lan, et al., Front. Biosci., 22:2 (2017); Liu, et al., Anticancer Res., 32:5 (2012). A recent systematic review shows that metformin use is associated with overall decrease in cancer incidence, even after adjusting for BMI and study biases. Gandini, et al., Canc. Prev. Res., 7:9 (2014). These results indicate that metformin’s mechanisms of action are different from the other insulin-based diabetes treatments, since they are usually associated with increased cancer risk. Currie, et al., Diabetologia, 52:9 (2009).

While some evidence suggests metformin can potentially be used to treat cancer, little is known about how metformin functions in the treatment of cancer. Further, the direct effect of metformin on T cells remains controversial. Some reports show that metformin can decrease T cell viability and increase apoptosis during homeostasis (see Solano, et al., Clin. Exp. Imm., 153:2 (2008)), increase the number of regulatory T cells and diminish the IL-17 producing Th17 cells in experimental autoimmune encephalomyelitis (see Sun, et al., J. Neuroimm., 292:58-67 (2016)). Very little is known about how metformin affects outcomes of new, modern cancer therapeutics in part because anti-cancer immune responses are extensively complicated and difficult to predict.

### SUMMARY

The compositions and methods disclosed herein address certain unmet needs in the cancer field. Tumor Membrane Vesicle (TMV) combination immunotherapies disclosed herein provide personalized approaches to treating cancers such as TNBC, which suffers from a dearth of effective personalized therapies. Despite failure of numerous known anti-cancer agents to provide positive therapeutic outcomes for TNBC patients in particular, the methods to treat cancers such as TNBC using TMV immunotherapy disclosed herein result in surprisingly effective treatments with significant therapeutic outcomes. The disclosed TMV + metformin combination therapies surprisingly inhibit primary tumor growth, metastatic spread, and infiltration of myeloid derived suppressor cell (MDSC). Further, TMV + metformin combination therapies surprisingly improved overall survival better than either treatment alone, to a degree that is greater than the effect of either treatment alone or the sum of their separate effects. Therefore, the present invention shows that TMV and MET synergize to inhibit tumor growth, metastatic spread, immune cell infiltration and/or to improve survival.

Disclosed herein are methods for treating a subject having, or at risk of having, a cancer, comprising administering to the subject a therapeutically effective amount of a tumor membrane vesicle (TMV) and a metformin. In some embodiments, the TMV comprises a lipid membrane, and a B7-1 and/or IL-12 molecule anchored to the lipid membrane. In some embodiments, the methods can further comprise administering an immune checkpoint inhibitor (e.g., an anti-CTLA4, anti-PD1, or anti-PD-L1 antibody). In some embodiments, the cancer is a breast cancer, for instance a triple-negative breast cancer (TNBC). The methods can reduce metastasis of the cancer, reduce the size of the tumor, reduces the amount of myeloid-derived suppressor cells (MDSCs) in the blood/serum, and/or increase the amount of pro-inflammatory cytokine production in a tumor. In some embodiments, the method is performed after surgical resection of a tumor. In some embodiments, the TMV, the metformin, or both are administered in two or more doses.

### BRIEF DESCRIPTION OF THE DRAWINGS

FIGS. 1(A-C) is a set of graphs showing tumor growth over time. FIG. 1A shows that mice were challenged with 4T1 murine breast cancer tumor cells, then treated with PBS, Metformin (MET) alone, TMVs derived from the 4T1 tumor cells, or a combination of TMV + MET. Tumor growth was measured over time. FIGS. 1(B-C) shows that combination of TMV vaccine and Metformin inhibits the growth of early and established 4T1 and SCC VII tumors. FIG. 1B shows that C3H/Hej mice were inoculated with 50,000 squamous cell carcinoma SCC VII cells s.c. in the hind flank. Tumors were palpable at day 3, which is when treatment with PBS, MET alone, TMV alone, or TMV + MET was initiated. The subsequent treatments were employed at days 10, 17, 24, and 40. FIG. 1C shows that C3H/Hej mice were inoculated with 50,000 SCC VII cells s.c. in the hind flank. Tumors were palpable at day 3, treatment of PBS control, MET alone, TMV alone, or TMV + MET was applied at days 10, 17, 24, 31, and 40. Tumor growth was measured over time. n=5 per group, two-way ANOVA with Tukey’s multiple comparison for statistical significance. For FIGS. 1(A-C), filled circle is PBS treatment group, filled square is MET alone treatment group, filled grey triangle is TMV alone treatment group, upside down triangle is MET + TMV treatment group. *p<0.05, **p<0.01, ***p<0.001.

FIGS. 2(A-C) is a set of graphs showing percentages of T-cell subtypes in the blood of mice with 4T1 tumor. After 4T1 tumor challenge and treatment with water, TMV alone, MET alone, or TMV+MET, mouse blood samples were analyzed for percentage of CD3+ (FIG. 2A), CD4+ (FIG. 2B), and CD8+ (FIG. 2C) T-cells, in relation to CD45+ T cells. Filled circle is water treatment group, filled square is MET treatment group, filled grey triangle is TMV treatment group, upside down triangle is TMV + MET treatment group.

FIGS. 3(A-B) is a set of Fluorescence Activated Cell Sorting (FACS) plots showing presence of myeloid-derived suppressor cells (MDSCs) (FIG. 3A) and CD3+ cells (FIG. 3B) in the blood of 4T1 tumor-challenged mice.

FIG. 4 is a set of graphs showing amounts of MDSCs (top panel) and CD3+ T cells (bottom panel) in the blood of 4T1 tumor-challenged mice treated with water, TMV alone, MET alone, or TMV+MET. Filled circle is water treatment group, filled square is MET treatment group, filled grey triangle is TMV treatment group, upside down triangle is TMV + MET treatment group. **p<0.01.

FIGS. 5(A-B) is a set of graphs showing amounts of tumor-infiltrating CD3+ (FIG. 5A) or CD8+ (FIG. 5B) T-cells in 4T1 tumor-challenged mice treated with PBS, TMV alone, MET alone, or TMV+MET. **p<0.01, ****p<0.0001.

FIGS. 6(A-B) is a set of graphs showing the amount of 4T1 tumor cells (FIG. 6A) and metastatic clones (FIG. 6B) obtained in the lungs of 4T1 tumor-challenged mice treated with water, TMV alone, MET alone, or TMV + MET. **p<0.01, ***p<0.001,

FIG. 7 is a graph showing a profile of pro-inflammatory cytokines produced in 4T1 tumors of mice treated with PBS, TMV alone, MET alone, or TMV+MET. CD8+ T-cells in tumors were analyzed for production of IFN-gamma, TNF-alpha, and IL-2 and plotted according to producers of one, two, or all three of these cytokines. Each column represents a subpopulation of CD8+ T cells according to their cytokine production profiles as shown in the table. Columns are separated by dash-dotted lines. Filled circle is PBS treatment group, filled square is MET alone treatment group, filled grey triangle is TMV alone treatment group, upside down triangle is MET + TMV treatment group. p<0.05, **p<0.01, ***p<0.001, ns is not significant.

FIG. 8 is a graph showing survival of mice treated with PBS, TMV alone, MET alone, or TMV+MET after surgical resection of tumors. Mice were challenged 10 days before surgery with 4T1 tumor cells. After tumor establishment, tumors were resected on Day 0. Beginning on Day 5, 50 mg/kg metformin was administered. On Days 5 and 12, 4T1-derived TMVs were administered. PBS treatment resulted in 0% survival at day 30; MET alone treatment resulted in 0% survival at day 60; TMV alone treatment resulted in 40% survival by day 100; and MET+TMV treatment resulted 60% survival by day 100. Solid line is PBS treatment group, dashed line is MET alone, dotted line is TMV alone, dash and dot line is TMV and MET treatment.

FIGS. 9(A-B) is a set of graphs showing combination of TMV vaccine and Metformin inhibits MOC2 tumor growth. C57BL/6J mice were inoculated with 500,000 oral squamous cell carcinoma MOC2 cells s.c. in the hind flank. Tumors were palpable at day 3 post challenge. PBS, MET alone, TMV alone, or a combination of TMV + MET was employed at days 3, 8, 13, 18, and 25. Tumor growth was measured over time. FIG. 9A shows mean tumor sizes in MET alone, TMV alone, or MET + TMV treatment groups in comparison to PBS treatment controls. FIG. 9B shows tumor sizes of the individual animals that received MET alone (left panel), TMV alone (middle panel), or combination of MET and TMV (right panel) in comparison to individuals that received PBS treatment. Filled circle is PBS treatment group, filled square is MET alone treatment group, filled grey triangle is TMV alone treatment group, upside down triangle is MET + TMV treatment group. n=5 per group, two-way ANOVA with Tukey’s multiple comparison for statistical significance. *p<0.05, **p<0.01.

FIG. 10 is a graph showing that TMV vaccine and Metformin synergize to inhibit spontaneous MOC2 lung metastasis. C57BL/6J mice were inoculated with 500,000 MOC 2 cells s.c. in the hind flank. Tumors were palpable at day 3 post challenge. PBS, MET alone, TMV alone, or a combination of TMV + MET was employed at days 3, 8, 13, 18, and 25. Lungs were harvested and dispersed into single cell suspension after 25 days of tumor growth. Metastatic tumor cells were selected using G418 in vitro for 11-14 days and metastatic cells were quantified using a Nexelcom T4 cell counter.

FIG. 11 is a graph showing that Metformin and TMV vaccination in combination with PD-1 blockade provides optimal protection from 4T1 tumor growth. Balb/c mice were challenged with 50,000 mammary carcinoma 4T1 cells and therapy was started 3 days after challenge when the tumors were palpable. TMV vaccine, Metformin was administered starting at day 3 and given every other day until the end of the experiment, TMV vaccines were administered at days 3 and 10, and PD-1 blockade were administered at days 4, 7, 12 and 15. Tumor growth was measured over time. Filled circle is PBS treatment group, filled square is MET alone treatment group, filled grey triangle is TMV alone treatment group, upside down triangle is PD-1 blockade treatment group, filled black triangle is TMV+MET+PD-1 blockade treatment group. n=5 mice per group.

### DETAILED DESCRIPTION

Described herein are methods for treating a subject having, or at risk of having, a cancer, comprising administering to the subject a therapeutically effective amount of a tumor membrane vesicle (TMV) and a metformin. It is a surprising finding of the present invention that the combination treatment comprising a TMV and a metformin results in a synergistic activity and improved cancer treatment.

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Terminology

Unless defined otherwise, all technical and scientific terms used herein have the same meaning as commonly understood to one of ordinary skill in the art to which this invention belongs. The following definitions are provided for the full understanding of terms used in this specification.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular therapeutic is disclosed and discussed and a number of modifications that can be made to the therapeutic are discussed, specifically contemplated is each and every combination and permutation of the therapeutic and the modifications that are possible unless specifically indicated to the contrary. Thus, if a class of therapeutics A, B, and C are disclosed as well as a class of therapeutics D, E, and F and an example of a combination therapeutic, or, for example, a combination therapeutic comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods. It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” or “administering” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject’s body (e.g. greater than 50% of the body), for example through entrance into the circulatory or lymphatic systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject’s body. Administration includes self-administration and the administration by another.

As used herein, the term “anchored to the lipid membrane” refers to the insertion of an exogenous polypeptide such as B7-1, B7-2 and/or IL-12 at the exterior of the lipid membrane surface. The term “anchored to the lipid membrane” does not refer to endogenous polypeptides naturally expressed at a cell’s surface. As a non-limiting example, a B7-1 exogenous polypeptide can be engineered to contain an amino acid sequence comprising a GPI membrane-anchor signal (referred to as “GPI-B7-1”). The GPI anchor sequence of such a polypeptide can insert into a natural or artificial lipid membrane, thereby “anchoring” the polypeptide to the lipid membrane.

“Pharmaceutically acceptable” component can refer to a component that is not biologically or otherwise undesirable, e.g., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics.

“Therapeutically effective amount” or “therapeutically effective dose” of a composition (e.g. a composition comprising an agent) refers to an amount that is effective to achieve a desired therapeutic result. In some embodiments, a desired therapeutic result is the reduction in tumor size or metastasis. Therapeutically effective amounts of a given agent will typically vary with respect to factors such as the type and severity of the disorder or disease being treated and the age, gender, weight, and general condition of the subject. Thus, it is not always possible to specify a quantified “therapeutically effective amount.” However, an appropriate “therapeutically effective amount” in any subject case may be determined by one of ordinary skill in the art using routine experimentation. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. It is understood that, unless specifically stated otherwise, a “therapeutically effective amount” of a therapeutic agent can also refer to an amount that is a prophylactically effective amount. In some instances, a desired biological or medical response is achieved following administration of multiple dosages of the composition to the subject over a period of days, weeks, or years.

“Treat,” “treating,” “treatment,” and grammatical variations thereof as used herein, include the administration of a composition with the intent or purpose of partially or completely, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease.

“Specifically binds” when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

### Tumor Membrane Vesicles (TMVs)

The tumor membrane vesicles (TMVs) used in the methods herein are described in PCT Patent Application PCT/US2013/024355 (WO2013/116656), U.S. Pat. No. 6,491,925 and in U.S. Pat. Application Publication US2018/0128833, the entire contents of which are herein incorporated by reference.

The TMV is cell membrane vesicle isolated from tumor tissue or an artificial or engineered particle containing cell membrane material obtained from a tumor (e.g., surgically resected patient tumor tissue). Tumor cell membrane material is formed on the surface of the particle, thereby exposing the cell membrane material to the external environment of the TMV. A TMV can be in the form of numerous particle types, including polymeric (synthetic or natural) nanoparticle, liposome, micelle, solid lipid nanoparticle (SLN), emulsions, and other particles capable of incorporating cell membrane material.

Because the TMV contains tumor cell membrane material, the TMV can contain tumor associated molecules and/or tumor-specific molecules (e.g., antigens). These tumor-specific antigens can activate the subject’s immune system by active immunization with tumor antigens. Thus, TMVs represent a personalized, tissue-derived strategy for treating tumors in a subject.

The TMV contains a lipid membrane which can comprise tumor associated molecules and/or tumor specific molecules (e.g., antigens). Further, additional molecules not specifically derived from a tumor or tumor sample can be attached to the lipid membrane. For instance, additional molecules can be inserted into the membrane (e.g., by hydrophobic interaction or by GPI-anchor) or can be tethered by covalent or ionic linkage to another molecule inserted into the membrane (e.g., covalent linkage to a polyethylene glycol molecule or a lipid molecule). These additional molecules can include one or more immunostimulatory agents, one or more antigens, and one or more additional anti-tumor compounds (e.g., anti-neoplastic agent). The lipid membrane may be in the form of a monolayer or bilayer (e.g., a phospholipid monolayer or phospholipid bilayer), or mixtures thereof, and can cover the entire surface or a portion of the surface of the TMV.

Typically, the TMV contains an immunostimulatory agent (ISM) attached to the lipid membrane of the TMV. As used herein, an “immunostimulatory agent” is any molecule that, when attached to a TMV, can stimulate or co-stimulate an anti-tumor immune response. TMVs containing membrane-attached immunostimulatory agents deliver molecules which stimulate immune responses, as well as patient-specific tumor antigens, and activate immune cells to promote an anti-tumor immune response. In some embodiments, the immunostimulatory agent is selected from the group consisting of CD7, B7-1 (CD80), B7-2 (CD86), 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6. ILT3, ILT4, and HVEM, and afunctional fragment thereof. In some embodiments, the immunostimulatory agent is B7-1 (also known as CD80), B7-2 (also known as CD86), IL-12, GM-CSF, IL-2 or combinations thereof. In some embodiments, the immunostimulatory agent is B7-1, B7-2, IL-12, or combinations thereof. In some embodiments, the immunostimulatory agent is B7-1, IL-12, or combinations thereof. In some embodiments, the TMV includes one immunostimulatory agent or, alternatively, two or more immunostimulatory agents. In some embodiments, the immunostimulatory agent is B7-1. In some embodiments, the immunostimulatory agent is B7-2. In some embodiments, the immunostimulatory agent is IL-12. In some embodiments, the immunostimulatory agent is GM-CSF. In some embodiments, the immunostimulatory agent is IL-2.

In some embodiments, the immunostimulatory agent B7-1 comprises an amino acid sequence of SEQ ID NO: 1 or a fragment thereof. In some embodiments, the immunostimulatory agent B7-1 comprises an amino acid sequence of SEQ ID NO: 2 or a fragment thereof. In some embodiments, the immunostimulatory agent B7-1 comprises an amino acid sequence of SEQ ID NO: 3 or a fragment thereof. In some embodiments, the immunostimulatory agent B7-1 is that identified in one or more publicly available databases as follows: HGNC: 1700 Entrez Gene: 941 Ensembl: ENSG00000121594 OMIM: 112203 UniProtKB: P33681. In some embodiments, the immunostimulatory agent B7-1 comprises a polypeptide sequence having about 70% or greater, about 75% or greater, about 80% or greater, about 85% or greater, about 90% or greater, about 95% or greater, or about 98% or greater homology with SEQ ID NO: 1, SEQ ID NO:2, or SEQ ID NO:3.

In some embodiments, the immunostimulatory agent B7-2 comprises the amino acid sequence of SEQ ID NO:4, or a fragment thereof. In some embodiments, the immunostimulatory agent B7-2 is that identified in one or more publicly available databases as follows: HGNC: 1705 Entrez Gene: 942 Ensembl: ENSG00000114013 OMIM: 601020 UniProtKB: P42081. In some embodiments, the immunostimulatory agent B7-2 used comprises a polypeptide sequence having about 70% or greater, about 75% or greater, about 80% or greater, about 85% or greater, about 90% or greater, about 95% or greater, or about 98% or greater homology with SEQ ID NO: 4.

In some embodiments, IL-12 comprises IL-12a and IL-12b. In some embodiments, the immunostimulatory agent IL-12 comprises the sequence of SEQ ID NO: 5, or a fragment thereof. In some embodiments, the immunostimulatory agent IL-12 is that found in one or more publicly available databases as follows: HGNC: 5969 Entrez Gene: 3592 Ensembl: ENSG00000168811 OMIM: 161560 UniProtKB: P29459. In some embodiments, the immunostimulatory agent IL-12 comprises a polypeptide sequence having about 70% or greater, about 75% or greater, about 80% or greater, about 85% or greater, about 90% or greater, about 95% or greater, or about 98% or greater homology with SEQ ID NO: 5.

In some embodiments, the immunostimulatory agent IL-12 comprises the sequence of SEQ ID NO: 6, or a fragment thereof. In some embodiments, the immunostimulatory agent IL-12 is that found in one or more publicly available databases as follows: HGNC: 5970 Entrez Gene: 3593 Ensembl: ENSG000001 13302 OMIM: 161561 UniProtKB P29460. In some embodiments, the immunostimulatory agent IL-12 comprises a polypeptide sequence having about 70% or greater, about 75% or greater, about 80% or greater, about 85% or greater, about 90% or greater, about 95% or greater, or about 98% or greater homology with SEQ ID NO: 6.

The immunostimulatory agent (e.g., B7-1, B7-2 and/or IL-12) is anchored to the lipid membrane of the vesicle. Other molecules, for instance, an antigen molecule such as a tumor specific antigen or cancer marker, can also be anchored to the lipid membrane of the vesicle. As used herein, the term “anchored to the lipid membrane” refers to the insertion of an exogenous polypeptide such as B7-1, B7-2 and/or IL-12 at the exterior of the lipid membrane surface. The term “anchored to the lipid membrane” does not refer to endogenous polypeptides naturally expressed at a cell’s surface.

In some embodiments, the immunostimulatory molecule (e.g., B7-1, B7-2 and/or IL-12), antigen molecule, or other molecules (e.g., tumor-specific proteins) can be anchored onto the membrane of the TMV through a variety of linkages, such as lipid palmatic acid, biotin-avidin interaction, or a glycosylphosphatidylinositol (GPI)-anchor. Accordingly, polypeptides described herein can be anchored to a lipid membrane, or TMV membrane via a glycosylphosphatidylinositol (GPI)-anchor. For example, glycosyl phosphatidylinositol anchored B7-1 (GPI-B7-1) molecules have been incorporated onto tumor cells and isolated tumor cell membranes to provide costimulation for allogenic T cell proliferation. See Nagarajan et. al., Vaccine, 24(13):2264-74 (2006), U.S. Published Pat. Application No. US 2007/0243159, U.S. Pat. No. 6,491,925, Bozeman et al., Front Biosci., 15:309-320 (2010), all incorporated by reference herein in their entireties. As used herein, a GPI-anchored molecule (for instance, B7-1) is preceded by “GPI-” (e.g., GPI-B7-1).

GPI-anchored polypeptides can be created through the addition of a GPI anchor signal sequence to the polypeptide. A GPI anchor signal sequence is a sequence that directs GPI anchor addition to the polypeptide. Over 150 different human polypeptides contain GPI anchors. See Kinoshita et. al., J. Lipid Res., 57(1):6-24 (2016). One example of a GPI anchor signal sequence that may be added to a polypeptide is SEQ ID NO: 11, a CD59 GPI anchor signal sequence. In some embodiments, the GPI anchor signal sequence comprises the sequence of SEQ ID NO: 11, or a polypeptide sequence having at or greater than about 80%, about 85%, about 90%, about 95%, or about 98% homology with SEQ ID NO: 11, or a polypeptide comprising a portion of SEQ ID NO: 11. Examples of other polypeptides containing a GPI signal sequence include, but are not limited to, 5′nucleotidease, CD48, FcgammaRIIIb, GPIHBP1, CD55. Accordingly, in some embodiments, the immunostimulatory agent, antigen, or other molecules attached to the lipid membrane include a GPI anchor signal sequence. Methods for creating GPI-anchored polypeptides are known in the art. See, e.g., U.S. Pat. No. 6,491,925, incorporated by reference herein in its entirety.

A number of proteins commonly expressed by cells are attached to the cell membrane via a GPI-anchor. These proteins are post-translationally modified at their carboxy terminus to express this glycosylated moiety which is synthesized in the endoplasmic reticulum. These naturally expressing GPI-anchored molecules are widely distributed in mammalian cells and serve a host of different cellular functions, such as cell adhesion, enzymatic activity, and complement cascade regulation. Naturally occurring GPI- anchored proteins lack a transmembrane and cytoplasmic domain that otherwise anchor membrane proteins. The GPI-anchor consists of a glycosylated moiety attached to phosphatidylinositol containing two fatty acids. The phosphatidylinositol portion, as well as an ethanolamine which is attached to the C-terminal of the extracellular domain of the membrane proteins, anchor the molecule to the cell membrane lipid bilayer.

To exploit this natural linkage using recombinant DNA techniques, the transmembrane and cytoplasmic domains of a transmembrane surface protein need only be replaced by the signal sequence for GPI-anchor attachment that is found at the hydrophobic C-terminus of GPI-anchored protein precursors. This method may be used to generate GPI- anchored proteins is not limited to membrane proteins; attaching a GPI-anchor signal sequence to a secretory protein also converts the secretory protein to a GPI-anchored form. The method of incorporating the GPI-anchored proteins onto isolated cell surfaces or TMVs is referred to here as protein transfer.

GPI-anchored molecules can be incorporated onto lipid membranes spontaneously. GPI-anchored proteins can be purified from one cell type and incorporated onto cell membranes of a different cell type. GPI-anchored proteins can be used to customize the lipid membranes disclosed herein. Multiple GPI-anchored molecules can be simultaneously incorporated onto the same cell membrane. The amount of protein attached to the TMV can be controlled by simply varying the concentration of the GPI-anchored molecules to be incorporated onto membranes. A significant advantage of this technology is the reduction of time in preparing cancer vaccines from months to hours. These features make the protein transfer approach a more viable choice for the development of cancer vaccines for clinical settings. The molecules incorporated by means of protein transfer retain their functions associated with the extracellular domain of the native protein. Cells and isolated membranes can be modified to express immunostimulatory agents. In certain embodiments, the disclosure contemplates that the GPI-anchored molecules are incorporated onto the surface of TMVs by this protein transfer method. GPI-anchored proteins attached to the surface of TMVs are used for an array of functions, at least including immunostimulation, co-stimulation, boosting immune responses, generating long term memory, etc., thereby enhancing the capacity to function as a targeted therapy for cancer treatment.

GPI-B7-1 incorporation (by protein transfer) was stable up to 7 days on isolated membranes at 37° C., and frozen membranes can be used up to 3 years of storage at -80° C., rendering stability and storage a nonissue. These studies show that membrane-based TMV vaccines are more suitable to stably express GPI-anchored molecules than intact cells, which significantly lose expression of the GPI-anchored molecules within about 24 hours.

The protein transfer strategy provides advantages over other immunotherapies for cancer vaccine development. Protein transfer allows a protein to be added either singularly or in a combinatory manner to the TMV surface. This approach does not require the establishment of tumor cells, unlike for gene transfer. This GPI-mediated approach by protein transfer may be used for an array of molecules, such as immunostimulatory agents (e.g., B7-1, B7-2, GM-CSF, IL-2, and IL-12), Further, immunostimulatory agents attached to the TMV via a GPI-anchor can exert their effector functions locally at the vaccination site with reduced or no risk of systemic toxicity.

In some embodiments, the TMV further comprises an antigen molecule. The antigen molecule can be attached to the lipid membrane of the TMV, for example by a GPI anchor. Thus, in some embodiments, the antigen molecule is modified to include a GPI-anchor amino acid sequence.

In some embodiments, the TMV further comprises two or more antigen molecules. For example, the TMV can comprise at least two, at least three, at least four, at least five, at least six, at least seven, at least eight, at least nine, at least ten, or more antigen molecules.

In some embodiments, the antigen molecule in the tumor membrane vesicle (TMV) can be HER-2, MKI67, prostatic acid phosphatase (PAP), prostate-specific antigen (PSA), prostate-specific membrane antigen, early prostate cancer antigen, early prostate cancer antigen-2 (EPCA-2), BCL-2, MAGE antigens such as CT7, MAGE- A3 and MAGE-A4, ER 5, G-protein coupled estrogen receptor 1, CA15-3, CA19-9, CA 72-4, CA-125, carcinoembryonic antigen, CD20, CD31, CD34, PTPRC (CD45), CD99, CD 117, melanoma-associated antigen (TA-90), peripheral myelin protein 22 (PMP22), epithelial membrane proteins (EMP-1, -2, and -3), HMB-45 antigen, MART-1 (Melan-A), S100A1, S100B and gp 100:209-217(210 M), MUC-1, mucin antigens TF, Tn, STn, glycolipid globo H antigen, or any combination thereof. Typically, the antigen is the human form. HER-2, or Human Epidermal Growth Factor Receptor 2, refers to the human protein encoded by the ERBB2 gene that has been referred to as Neu, ErbB-2, CD340 (cluster of differentiation 340) or pl85. See Coussens et al, Science, 230 (4730): 1132-9 (1985).

In some embodiments, the antigen molecule comprises HER-2 or a fragment thereof. In some embodiments, the HER-2 comprises an amino acid of SEQ ID NO: 7 or a fragment thereof. In some embodiments, the antigen molecule HER-2 comprises an amino acid sequence identical to SEQ ID NO: 8 or a fragment thereof. In some embodiments, the antigen molecule HER-2 comprises an amino acid sequence of, SEQ ID NO: 9 or a fragment thereof. In some embodiments, the antigen molecule HER-2 is that identified in one or more publicly available databases as follows: HGNC: 3430 Entrez Gene: 2064 Ensembl: ENSG00000141736 OMIM: 164870 UniProtKB: P04626. In some embodiments, the antigen molecule HER-2 comprises a polypeptide sequence having about 70% or greater, about 75% or greater, about 80% or greater, about 85% or greater, about 90% or greater, about 95% or greater, or about 98% or greater homology with SEQ ID NO: 7, SEQ ID NO: 8, or SEQ ID NO: 9.

In some embodiments, the antigen molecule is or comprises a HER-2 or a fragment thereof. In some embodiments the antigen molecule is or comprises a PAP or a fragment thereof. In some embodiments, the antigen molecule is or comprises a PSA or a fragment thereof.

### Metformin

The compositions and treatments disclosed herein include a metformin and/or a metformin derivative. Metformin has the molecular formula C₄H₁₁N₅. In some embodiments, the metformin is a 3-(diaminomethylidene)-1,1-dimethylguanidine. In some embodiments, the metformin has the chemical structure shown below (Formula I).

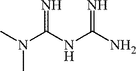

The term “metformin” includes all forms of the compound of Formula I, including hydrates, solvates, isomers, crystalline and non-crystalline forms, isomorphs, and polymorphs thereof. For example, the term “metformin” includes the metformin salt metformin hydrochloride. As used herein, the term “metformin derivative” includes the compositions described in U.S. Pat. Application Publication 2017/0247400 and U. S. Pat. Publication 2011/0257432, both of which are incorporated herein by reference. In some embodiments, the metformin derivative has the following chemical structure.

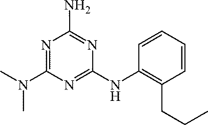 [SMILES: CCCc1ccccc1Nc1nc(N)nc(N(C)C)n1]

While some studies indicate metformin can be used in the treatment of cancer, other studies showed metformin can induce counter-productive effects in treating cancer. For instance, metformin can decrease T cell viability and increase apoptosis during homeostasis (see Solano, et al., Clin. Exp. Imm., 153:2 (2008)), increase the number of regulatory T cells and diminish the IL-17 producing Th17 cells in experimental autoimmune encephalomyelitis (see Sun, et al., J. Neuroimm., 292:58-67 (2016)). Such effects of Metformin indicate that the compound may function as suppressor for effector T cell responses.

The inventors herein, however, discovered that a combination therapy comprising a TMV and a metformin (MET) can be surprisingly beneficial in the treatment of cancer. For instance, while the administration of MET alone failed to suppress tumor growth or metastasis, the TMV + MET combination therapy can inhibit primary tumor growth, metastatic spread, and infiltration of myeloid derived suppressor cell (MDSC). Importantly, the administration of TMV alone only conferred a mild suppression on tumor growth, indicating that the increased suppression of tumor growth is due to a synergistic effect of TMV + MET rather than an additive effect. Further, TMV + metformin combination therapies surprisingly improved the overall survival rate better than either treatment alone, whereas the administration of MET failed to improve the survival rate at all. Therefore, the present invention shows that TMV and MET synergize to inhibit tumor growth, metastatic spread, immune cell infiltration and/or improve survival.

### Additional Agents

The compositions and methods described herein can further include the administration of an immune checkpoint inhibitor (ICI). Immune checkpoint inhibitors (sometimes referred to as checkpoint blockade inhibitors (CBI) or checkpoint inhibitors) can increase the effectiveness of overall T cell anti-tumor immunity. ICIs block certain activities of particular proteins produced by immune cells (e.g., T cells) and cancer cells that keep immune cells “in check,” or in other words, prevent immune cells from attacking or killing a cell (e.g., cancer cell). When ICIs block checkpoint proteins, immune cells such as T cells can more effectively mount a response to the cancer cell.

In some embodiments, the compositions and methods include an antibody, particularly an antibody having ICI function. In some embodiments, the compositions and methods include an anti-CTLA4 antibody, an anti-PD1 antibody, an anti-PDL1 antibody, an anti-PDL2, or any combination thereof. In some embodiments, the compositions and methods include an anti-CTLA4 antibody, an anti-PD1 antibody, an anti-PDL1 antibody, or any combination thereof. In some embodiments, the compositions and methods include an anti-CTLA4. In some embodiments, the compositions and methods include an anti-PD1. In some embodiments, the compositions and methods include an anti-PD-L1. In some embodiments, the compositions and methods include an anti-PD-L2.

In some embodiments, the anti-CTLA4 antibody comprises abatacept, belatacept, ipilimumab, tremelimumab, or any combination thereof. In some embodiments, the anti-CTLA4 antibody is ipilimumab. An anti-CTLA4 antibody is defined herein as a polypeptide capable of specifically binding a CTLA4 polypeptide. In some embodiments, the anti-PDL1 antibody comprises atezolizumab, durvalumab, avelumab, or any combination thereof. In some embodiments, the anti-PDL1 antibody is atezolizumab (MPDL3280A) (Roche), durvalumab (MEDI4736), avelumab (MS0010718C), or any combination thereof. An anti-PDL1 antibody is defined herein as a polypeptide capable of specifically binding a PDL1 polypeptide. In some embodiments, the anti-PD1 antibody is nivolumab, pembrolizumab, or any combination thereof. An anti-PD-1 antibody is defined herein as a polypeptide capable of specifically binding PD-1 polypeptide.

Also included herein are compositions and methods that include a programmed death protein 1 (PD-1) inhibitor, programmed death protein ligand 1 or 2 inhibitor, or any combination thereof. PD-1 inhibitors are known in the art, and include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech).

The compositions and treatments disclosed herein can also include one or more anti-neoplastic agents. In some embodiments, the anti-neoplastic agent can include Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPOX, Carboplatin, CARBOPLATIN-TAXOL, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Interferon Alfa-2b, Recombinant, Intron A (Recombinant Interferon Alfa-2b), Iodine I 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and Iodine I 131 Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Unituxin (Dinutuximab), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate), and combinations thereof.

### Methods for Treating

As discussed above, the present invention shows that TMV and MET surprisingly synergize to inhibit tumor growth, metastatic spread, immune cell infiltration and/or improve survival. Accordingly, disclosed herein is a method for treating a subject having, or at risk of having, a cancer, comprising administering to the subject a therapeutically effective amount of a tumor membrane vesicle (TMV) and a metformin. The TMV used in the methods can be any herein disclosed TMV.

The subject can be any mammalian subject, for example a human, dog, cow, horse, mouse, rabbit, etc. In some embodiments, the subject is a primate, particularly a human. The subject can be a male or female of any age, race, creed, ethnicity, socio-economic status, or other general classifiers.

The subject has, or is at risk of having, a cancer. Non-limiting examples of cancers include Acute granulocytic leukemia, Acute lymphocytic leukemia, Acute myelogenous leukemia (AML), Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma, Bile duct cancer, Bladder cancer, Bone cancer Bone marrow cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia (CLL), Chronic myelogenous leukemia (CML), Colon cancer, Colorectal cancer, Craniopharyngioma, Cutaneous lymphoma, Cutaneous melanoma, Diffuse astrocytoma, Ductal carcinoma in situ (DCIS), Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors (GIST), Germ cell tumor, Gestational Trophoblastic Disease (GTD), Glioblastoma multiforme (GBM), Glioma, Hairy cell leukemia, Head and neck cancer, Hemangioendothelioma, Hodgkin’s lymphoma, Hypopharyngeal cancer, Infiltrating ductal carcinoma (IDC), Infiltrating lobular carcinoma (ILC), Inflammatory breast cancer (IBC), Intestinal Cancer, Intrahepatic bile duct cancer, Invasive / infiltrating breast cancer, Islet cell cancer, Jaw/oral cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Mycosis Fungoides, Myelodysplastic Syndrome, Nasal cavity cancer, Nasopharyngeal cancer, Neck cancer, Neuroblastoma, Neuroendocrine tumors (NETs), Non-Hodgkin’s lymphoma, Non-small cell lung cancer (NSCLC), Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget’s disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system (CNS) lymphoma, Prostate cancer, Rectal cancer, Renal cell carcinoma, Renal pelvis cancer, Rhabdomyosarcoma, Salivary gland cancer, Sarcoma, Sinus cancer, Skin cancer, Small cell lung cancer (SCLC), Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma, Testicular cancer, Throat cancer, Thymoma / thymic carcinoma, Thyroid cancer, Tongue cancer, Tonsil cancer, Transitional cell cancer, Transitional cell cancer, Triple-negative breast cancer, Tubal cancer, Tubular carcinoma, Ureteral cancer, Urethral cancer, Uterine adenocarcinoma, Uterine cancer, Uterine sarcoma, Vaginal cancer, Vulvar cancer, Wilms tumor, Waldenstrom macroglobulinemia, etc., and combinations thereof.

A subject can be at risk of having a cancer by, for example, exposure to toxic-levels of a carcinogenic agent, exposure to chronic inflammation, infection with a cancer-causing microorganism, or by being genetically predisposed to having a cancer. For example, and without limitation, breast cancer genetic predisposition can arise from mutations in one or both alleles of BRCA1, BRCA2, CHEK2, ATM, BRIP1, PALB2, RAD50, RAD51B, RAD51C, RAD51D, XRCC2, CDH1, TP53, PTEN, STK11/LKB1, FGFR2, p53, NBS1, BARD1, MRE11, FANCA, FANCC, and FANCM, among other genetic biomarkers of breast cancer. A subject at risk of having a cancer includes a subject previously diagnosed with a cancer and subsequently clinically determined to be in partial or complete remission, and includes a subject previously diagnosed with a cancer that has undergone a procedure (e.g. surgery) to remove some or all of a cancer tumor.

In some embodiments, the methods and compositions disclosed herein can be used to treat a cancer that is selected from the group consisting of lymphoma, B cell lymphoma, T cell lymphoma, mycosis fungoides, Hodgkin’s Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma, glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers, testicular cancer, colon cancer, rectal cancer, prostatic cancer, and pancreatic cancer.

In some embodiments, the cancer comprises breast cancer. In some embodiments, the breast cancer can be triple-negative breast cancer. Triple negative breast cancer (TNBC) is defined as a cancer or tumor lacking expression of estrogen receptor, progesterone receptor, and HER-2 protein. TNBC represents one of the most challenging cancers for developing an effective therapy post tumor resection due to lack of a therapeutic target. Even with conventional radiation and chemotherapy regimens, patients can have poor prognosis, experiencing early, frequent relapses in comparison to other breast cancers. In addition, a high level of intratumoral as well as patient-to-patient heterogeneity is observed among triple negative patients, making it even more difficult to treat. See Gerlinger et. al., N. Engl. J. Med., 366:883-92 (2012). Therapies effective for other cancers, even other breast cancers, frequently prove ineffective at treating TNBC. Thus, it is difficult to predict therapeutic outcomes in TNBC for known anti-cancer agents and treatment regimens. TNBC is a clear area of significant unmet medical need, and new therapies that address patient-to-patient variation in tumor targets are critically required. See Peddi et. al., Int. J. Breast Cancer, 217185 (2012). In some embodiments, the triple negative breast cancer is a metastatic triple negative breast cancer.

In some embodiments, the cancer is squamous cell carcinoma. In some embodiments, the cancer is squamous cell carcinoma of head and neck. In some embodiments, the cancer is squamous cell carcinomas of the mouth. In some embodiments, the cancer is lung cancer.

The TMV used in the methods can comprise an immunostimulatory agent anchored to the lipid membrane. The immunostimulatory agent can be any herein disclosed immunostimulatory agent. In some embodiments, the immunostimulatory agent can comprise a full-length polypeptide or, alternatively, can comprise an immunostimulatory portion of full-length immunostimulatory agent.

In some embodiments, the immunostimulatory agent comprises a B7-1, B7-2, or IL-12 molecule. In some embodiments, the immunostimulatory agent comprises a B7-1 or IL-12 molecule. In some embodiments, only a B7-1 molecule is selected. In some embodiments, only a IL-12 molecule is selected. In some embodiments, the TMV comprises both a B7-1 and a IL-12 molecule anchored to the lipid membrane. In some embodiments, the TMV further comprises one or more additional immunostimulatory agents, for instance, B7-2, GM-CSF, and/or IL-2.

In some embodiments, the immunostimulatory agent can be anchored onto the membrane of the TMV through a variety of linkages, such as via lipid palmatic acid, biotin-avidin interaction, or a glycosylphosphatidylinositol (GPI)-anchor. In some embodiments, the GPI-anchored immunostimulatory agent (e.g., IL-12) has reduced liver toxicity as compared to the soluble form of the molecule.

In some embodiments, the TMV further comprises an antigen molecule anchored to the lipid membrane. The antigen molecule can comprise any herein disclosed antigen molecule. The entire antigen molecule or, alternatively, an antigenic portion of the antigen molecule can be used. In some embodiments, the antigen is a protein, or alternatively, an antigenic fragment of a protein. In some embodiments, the TMV contains an antigen molecule comprising HER-2, PSA, or PAP. Optionally, the antigen molecule is HER-2. In some embodiments, the antigen molecule is the extracellular domain of HER-2 which includes the peptide consisting essentially of amino acids 63-71 of human HER-2 (the “p63-71” peptide) having a sequence of SEQ ID NO: 10.

In some embodiments, the antigen molecule may be anchored onto the membrane of the TMV through a variety of linkages, such as via lipid palmatic acid, biotin-avidin interaction, or a glycosylphosphatidylinositol (GPI)-anchor.

In some embodiments, the method can further comprise administering to the subject a therapeutically effective amount of an additional agent selected from the group consisting of an anti-CTLA4 antibody, an anti-PD1 antibody, and an anti-PDL1 antibody, or any combination thereof. In some embodiments, the method can further comprise administering to the subject a therapeutically effective amount of an additional agent selected from the group consisting of an anti-CTLA4 antibody, an anti-PD1 antibody, an anti-PDL1 antibody, an anti-PDL2, or any combination thereof. In some embodiments, the method can further comprise administering to the subject a therapeutically effective amount of an anti-CTLA4 antibody. In some embodiments, the method can further comprise administering to the subject a therapeutically effective amount of an anti-PD1 antibody. In some embodiments, the method can further comprise administering to the subject a therapeutically effective amount of an anti-PD-L1 antibody. In some embodiments, the method can further comprise administering to the subject a therapeutically effective amount of an anti-PD-L2 antibody.

In some embodiments, the anti-CTLA4 antibody can include abatacept, belatacept, ipilimumab, tremelimumab, or any combination thereof. In some embodiments, the anti-CTLA4 antibody is ipilimumab. In some embodiments, the anti-PDL1 antibody can include atezolizumab, durvalumab, avelumab, or any combination thereof. In some embodiments, the anti-PDL1 antibody is atezolizumab (MPDL3280A) (Roche), durvalumab (MEDI4736), avelumab (MS0010718C), or any combination thereof. In some embodiments, the PD-1 inhibitor can include, for example, nivolumab (BMS), pembrolizumab (Merck), pidilizumab (CureTech/Teva), AMP-244 (Amplimmune/GSK), BMS-936559 (BMS), and MEDI4736 (Roche/Genentech). In some embodiments, the anti-PD1 antibody is nivolumab, pembrolizumab, or any combination thereof. In some embodiments, the administering step can include administration of an anti-neoplastic agent. The anti-neoplastic agent can be any herein disclosed anti-neoplastic agent.

In some embodiments, the method further comprises administering an adjuvant. The adjuvant can be administered prior to, concurrent with, or subsequent to administration of the TMV, or the metformin. In some embodiments, the adjuvant is GM-CSF, or any biocompatible FDA-approved adjuvant. In some embodiments, the adjuvant comprises IL-2, ICAM-1, GM-CSF, flagellin, unmethylated, CpG oligonucleotide, lipopolysaccharides, or lipid A. The adjuvant can be in a form separate from the TMV or can be anchored to the lipid membrane of the TMV (by, for example, via a GPI anchor). In some embodiments, the TMV further comprises an adjuvant anchored to the lipid membrane wherein the adjuvant and antigen molecule are not the same molecule.

The administering step can include any method of introducing the TMV and the metformin, separately or together, into the subject appropriate for the combination therapy formulation. The administering step can include at least one, two, three, four, five, six, seven, eight, nine, or at least ten dosages. The administering step can be performed before the subject exhibits disease symptoms (e.g., prophylactically), or during or after disease symptoms occur or after other treatment modalities such as surgery, chemotherapy, and radiation. The administering step can be performed prior to, concurrent with, or subsequent to administration of other agents to the subject. The administering step can be performed with or without co-administration of additional agents (e.g., additional immunostimulatory agents, anti-neoplastic agents).

The method can include systemic administration of the TMV and/or the metformin (e.g., injection into the circulatory or lymphatic systems). Alternatively, the method can include local administration of the TMV and/or the metformin. For example, the TMV and the metformin can be administered locally to a tumor or an area near a tumor. In some embodiments, the TMV and the metformin are administered to one or more areas of the subject comprising a tumor. Alternatively, the method can include systemic administration of the metformin and local administration of the TMV.

The disclosed methods can be performed any time prior to and/or after the onset of a cancer (e.g., a breast cancer or/and a squamous cell carcinoma). In some aspects, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years;12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of a cancer (e.g., a breast cancer or/and a squamous cell carcinoma); or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of a cancer (e.g., a breast cancer or/and a squamous cell carcinoma).

In some embodiments, a subsequent administration is provided at least one day after a prior administration, or at least two days, at least three days, at least four days, at least five days, or at least six days after a prior administration. In some embodiments, a subsequent administration is provided at least one week after a prior administration, or at least two weeks, at least three weeks, or at least four weeks after a prior administration. In some embodiments, a subsequent administration is provided at least one month, at least two months, at least three months, at least six months, or at least twelve months after a prior administration.

A dose of TMV and a dose of metformin can be administered together separately. Alternatively, the methods can comprise one or more separate doses of a TMV and a metformin, and one or more joint doses of a TMV and a metformin together. In some embodiments, the first dose of the TMV and the first dose of the metformin are administered after surgical resection of a tumor from the subject.

The amount of the disclosed compositions administered to a subject will vary from subject to subject, depending on the nature of the disclosed compositions and/or formulations, the species, gender, age, weight and general condition of the subject, the mode of administration, and the like. Effective dosages and schedules for administering the compositions may be determined empirically, and making such determinations is within the skill in the art. The dosage ranges for the administration of the disclosed compositions are those large enough to produce the desired effect (e.g., to reduce tumor size). The dosage should not be so large as to outweigh benefits by causing adverse side effects, such as unwanted cross-reactions, anaphylactic reactions, and the like, although some adverse side effects may be expected. The dosage can be adjusted by the individual clinician in the event of any counterindications. Generally, the disclosed compositions and/or formulations are administered to the subject at a dosage of active component(s) ranging from 0.1 µg/kg body weight to 100 g/kg body weight. In some embodiments, the disclosed compositions and/or formulations are administered to the subject at a dosage of active component(s) ranging from 1 µg/kg to 10 g/kg, from 10 µg/kg to 1 g/kg, from 10 µg/kg to 500 mg/kg, from 10 µg/kg to 100 mg/kg, from 10 µg/kg to 10 mg/kg, from 10 µg/kg to 1 mg/kg, from 10 µg/kg to 500 µg/kg, or from 10 µg/kg to 100 µg/kg body weight. Dosages above or below the range cited above may be administered to the individual subject if desired.

The disclosed methods can provide an array of therapeutic benefits when performed on a subject having a cancer. In some embodiments, the treatment comprising administering to a subject a therapeutically effective amount of a TMV and a metformin reduces metastasis of a cancer. In some embodiments, the treatment reduces the size of a tumor. In some embodiments, the treatment does not result in substantial liver toxicity. In some embodiments, the treatment reduces the amount of myeloid-derived suppressor cells in a biological sample (e.g., a blood or serum sample) of the subject. In some embodiments, the treatment increases the amount of CD8+ T-cells in a tumor. In some embodiments, the treatment increases the amount of pro-inflammatory cytokine (e.g., IFN-gamma, TNF-alpha, IL-2) production in a tumor.

The aforementioned therapeutic benefits can be determined by comparison to a control. The control can be a biological sample, for example a biological sample obtained from the subject prior to performing a disclosed method. Alternatively, a collection of values used as a standard applied to one or more subjects (e.g., a general number or average that is known and not identified in the method using a sample). In some embodiments, the control comprises a blood or serum sample obtained from the subject prior to the administration step (e.g., a baseline sample).

In some embodiments, the method can further include administering to the subject a therapeutically effective amount of an a TMV and a metformin, and a pharmaceutically acceptable excipient. Suitable excipients include, but are not limited to, salts, diluents, binders, fillers, solubilizers, disintegrants, preservatives, sorbents, and other components. Also disclosed herein is a medicament comprising a pharmaceutically effective amount of a TMV and a metformin.

In some embodiments, the method includes administering to the subject a medicament comprising a pharmaceutically effective amount of a TMV and a metformin. In some embodiments, the methods can further include administering to the subject a pharmaceutically effective amount of an immunostimulatory molecule, and/or an adjuvant. Generally, the medicament comprises a pharmaceutically acceptable excipient and a pharmaceutically effective amount of a metformin and a TMV.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is °C or is at ambient temperature, and pressure is at or near atmospheric. There are numerous variations and combinations of process conditions that can be used to optimize product quality and performance. Only reasonable and routine experimentation will be required to optimize such process conditions.

### Example 1. Tumor Membrane Vesicle (TMV) Immunotherapy and Metformin Combination Therapy to Treat Cancer

The use of tumor membrane vesicles (TMVs) permits a personalized immunotherapy platform that utilizes patient tumor tissue to encapsulate the antigenic landscape of each patient, which can enhance the immune response against the tumor. See Patel et al., Biomat., 74:231-244 (2016); Bhowmik et al., J. Microencapsul. 28:294-300 (2011); Bumgarner et al., J. Contrl. Release, 137:90-97 (2009); Nagarajan et al., Vaccine 24:2264-74 (2006). Protein Transfer can be used to anchor GPI-linked immunostimulatory molecules (ISMs) onto the lipid bilayer of TMVs, which can improve pro-inflammatory responses and anti-tumor responses elicited by TMV administration. See Bumgarner et al., J. Contrl. Release, 137:90-97 (2009); Bozeman et al., Vaccine, 31:2449-2456 (2013). GPI-anchored interleukin 12 (GPI-IL-12) and GPI-anchored B7-1 (GPI-B7-1) proteins can be used to induce immune responses in pre-clinical murine models.

TMV administration was combined with metformin treatment to study the effects of the combination therapy in mouse cancer models. Mice were divided into four groups of five mice per group, wherein each group was to be treated with either a PBS control, metformin (MET) alone (Metformin Hydrochloride, Sigma-Aldrich (Millipore Sigma), CAS Number 1115-70-4), TMV alone, or TMV+MET.

TMV were prepared and incorporated with GPI-proteins as previously described. See McHugh et. al., PNAS, 92:8059-63 (1995). Briefly, tumors were grown s.c. in the hind flanks and excised upon reaching 10 mm in diameter and frozen at -80° C. Tumors were then minced and homogenized using a disposable Omni tip homogenizer (Omni International, Kennesaw, GA) and centrifuged over a 41% sucrose gradient at 100,000 × g. TMV were collected from the interface, washed, and resuspended in PBS. TMV concentration was then determined using a micro BCA assay (Thermo Scientific, Rockford, IL). TMV were then incorporated with GPI-proteins at 2.5 µg/100 µg TMV for 4 h at 37° C. with gentle rotation, centrifuged, and resuspended in PBS prior to injection at 1 mg/ml final concentration. Incorporation of GPI-mB7-1 (anti-mouse CD80-APC, Clone 16-10A1) and GPImIL-12 (anti-mouse IL-12 p40-PE, Clone C17.8) was evaluated using flow cytometry (data not shown).

Each mouse was challenged with 4T1 mouse breast cancer cells by subcutaneous injection on Day 0. On Day 3, mice in the MET and TMV+MET groups were administered 50 mg/kg metformin in drinking water daily until Day 30. On Days 3 and 10, mice in the TMV and TMV+MET groups were administered a dose of TMVs prepared from 4T1 tumors. Mice were analyzed for tumor growth throughout the study period.

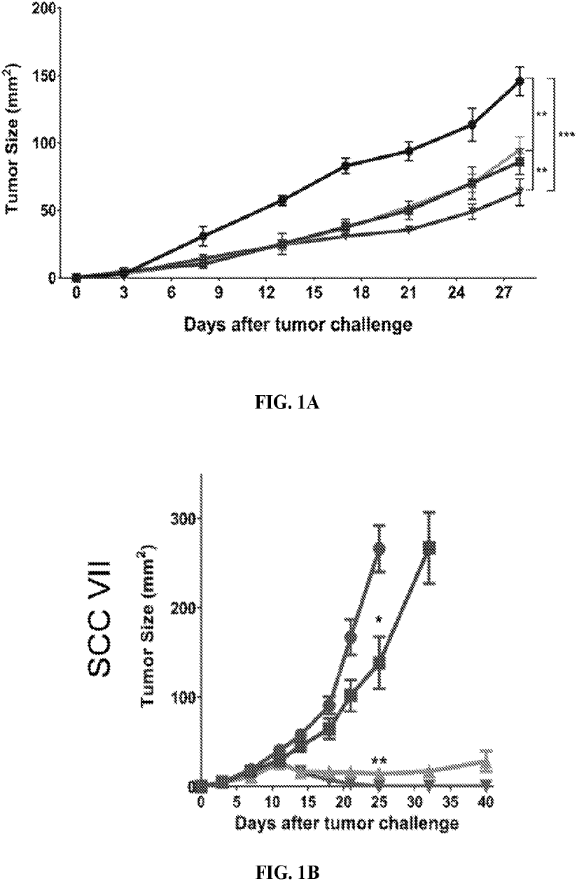

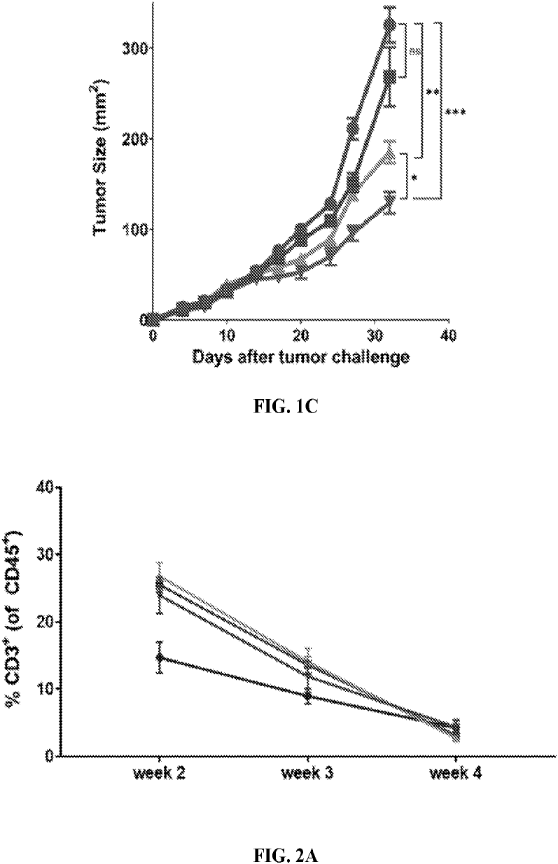

Tumors in untreated control mice grew to about 150 mm² by about 4 weeks (FIG. 1A). While treatments with MET alone and TMV alone significantly reduced tumor growth to below 100 mm² over the same period, the combination therapy TMV+MET reduced tumor growth more extensively than either treatment alone, resulting in tumors having a size of a little over 50 mm².

When the tumor challenge cells (and cells used to form TMVs) were substituted for a squamous cell tumor model cell line (SCCVII cells), significant reduction in tumor growth (about 250 mm²) was observed in TMV-treated mice (FIG. 1B). Both MET alone and TMV alone reduced tumor size; however the effect was much greater in the TMV-treated group than in the MET-treated group. As with the 4T1-challenged mice, the TMV+MET combination therapy reduced tumor size more extensively than either treatment alone. Unlike the 4T1-challenged mice, however, the SCCVII-challenged mice had essentially no observable tumors by Day 25, thereby showing the dramatic effect observed in a squamous cell cancer model using the TMV+MET combination therapy. In the same model (SCCVII), the TMV and MET treatment were delayed and given at Day 10 when the tumors were established (about 50 mm²) (FIG. 1C). Here, the SCCVII-challenged mice treated with MET did not have a significant decrease in growth, however the TMV treatment remained effective, although less so than when given at Day 3. The combination treatment had the smallest tumor sizes, showing the efficacy of combination therapy.

The 4T1-challenged mice were further analyzed for immunological effects upon administration of TMV, MET, or both. Blood samples were obtained from mice, and samples were subjected to analysis to determine presence of immune cell subtypes.

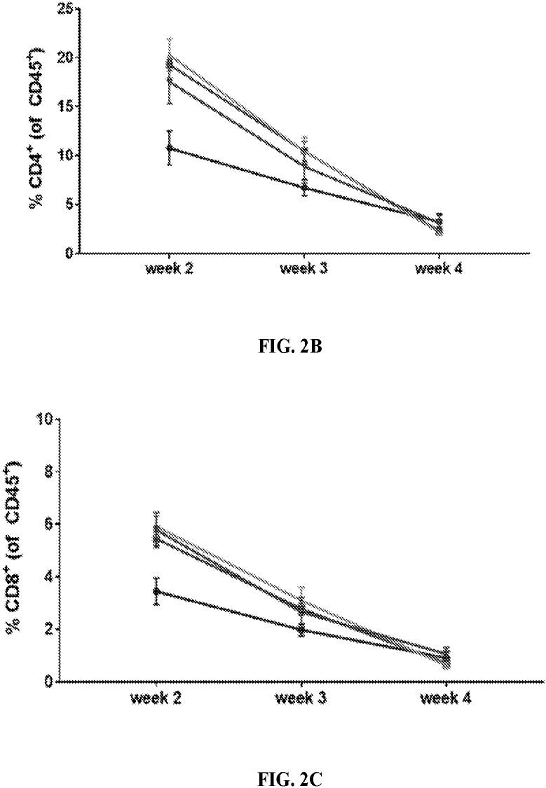

All three treatments (TMV, MET, TMV+MET) increased the amount of CD3+ (FIG. 2A), CD4+ (FIG. 2B), and CD8+ (FIG. 2C) T cells in the blood of treated mice by week 2, as compare to untreated PBS control mice. Results were not significant at other time points or between the treatment groups. These results show that CD3, CD4 and CD8 T cell percentages were higher in early stages in treated groups.

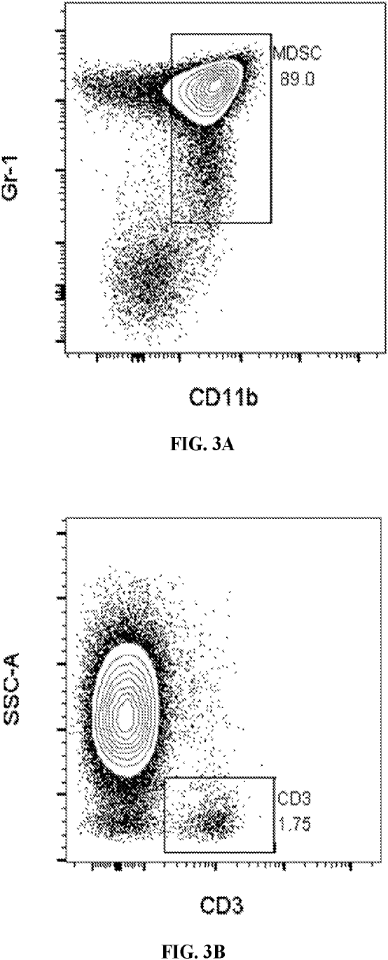

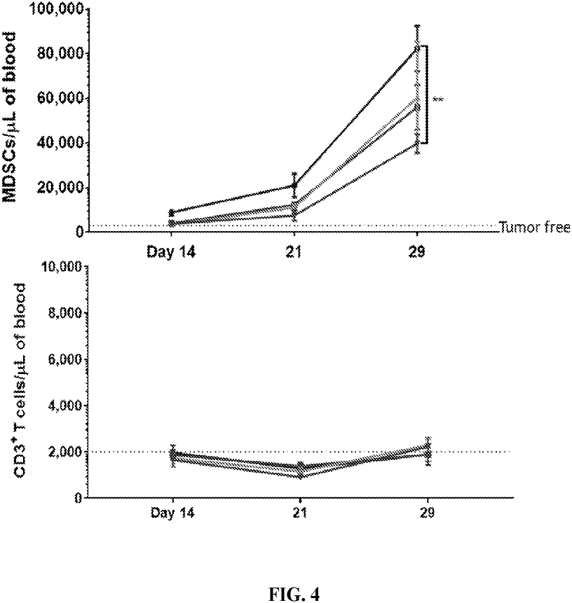

All three treatments (TMV, MET, TMV+MET) decreased the amount of myeloid derived suppressor cells (MDSC) in blood samples obtained from treated mice (FIG. 3A, FIG. 4 upper panel). However, MDSC reduction was more prominent in the TMV+MET combination therapy group than in either single treatment group.

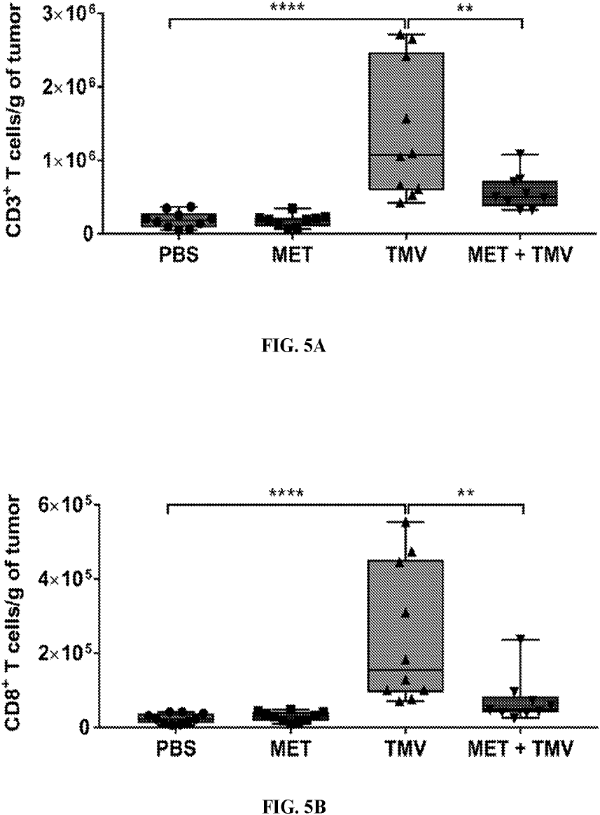

The TMV and TMV+MET treated groups also increased the amount of tumor-infiltrating CD3+ (FIG. 5A) and CD8+ T cells in biopsied tumor samples (FIG. 5B). Interestingly, while treatments with TMV+MET produced lower amounts of CD8+ T cell infiltration compared to treatment with TMV alone, the TMV+MET combination therapy reduced tumor growth more effectively than TMV treatments alone (See FIG. 1A).

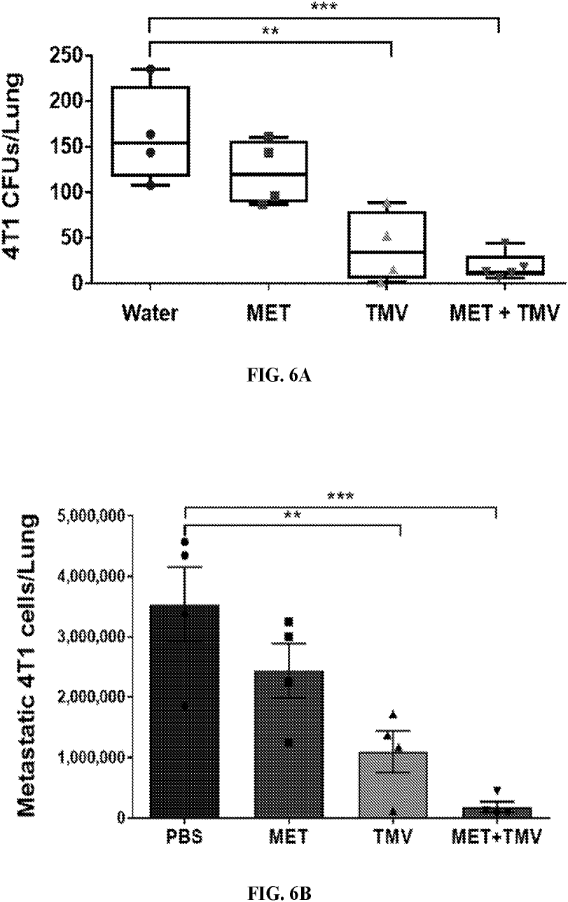

4T1-challenged mice were also analyzed for their metastatic potential. Upon sacrificing the mice, the lungs were analyzed for tumor colony forming units (FIG. 6A) and overall total tumor cells (FIG. 6B). In both instances, TMV alone but not MET alone significantly reduced the amount of metastasis observed in the lungs of mice. However, the TMV+MET combination therapy more extensively reduced metastasis compared to either treatment alone.

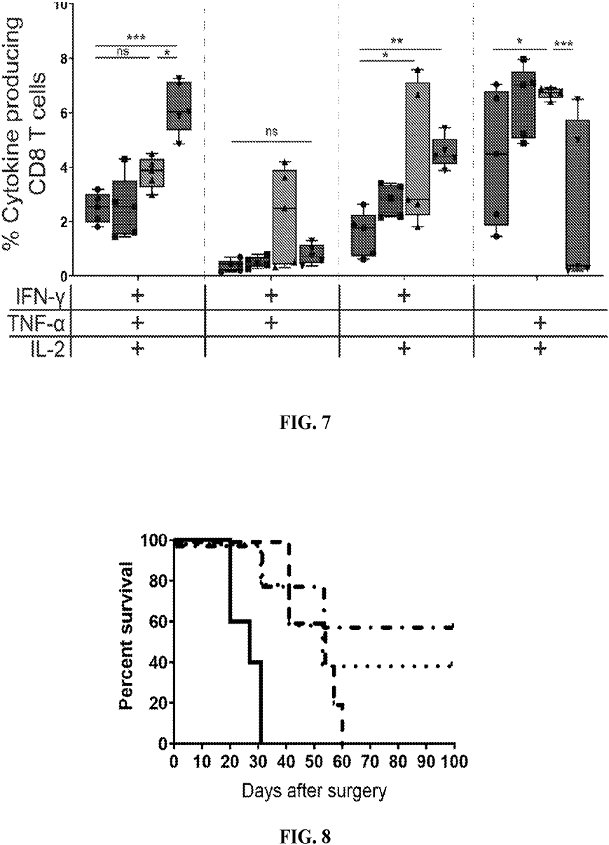

4T1-challenged mice were also analyzed for production of pro-inflammatory cytokines within the tumor environment. CD8+ T-cells were sorted, and were detected for production of IFN-gamma, TNF-alpha, and IL-2. The four treatment groups were categorized as producers of one, two, or all three of these tested cytokines (FIG. 7). Generally, treatment with the TMV+MET combination therapy resulted in increased production of IFN-gamma, TNF-alpha, and IL-2 within tumors.

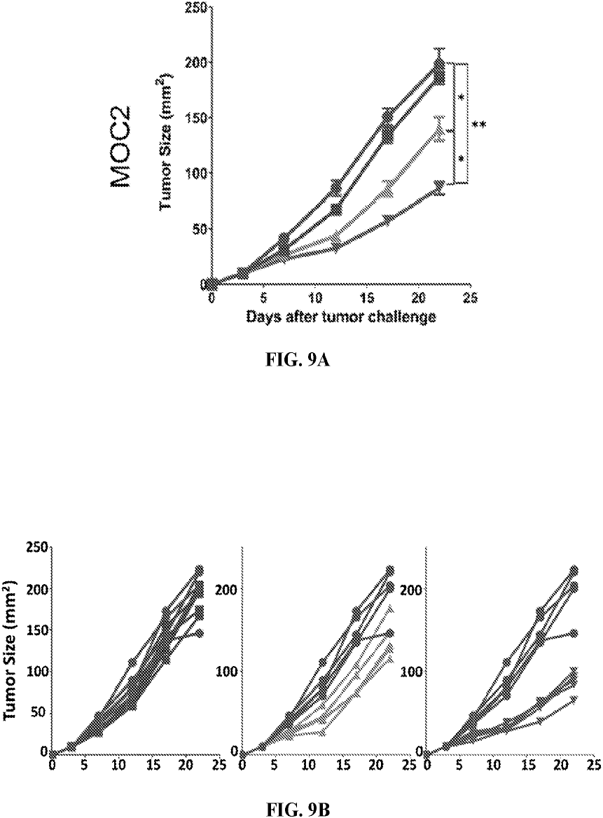

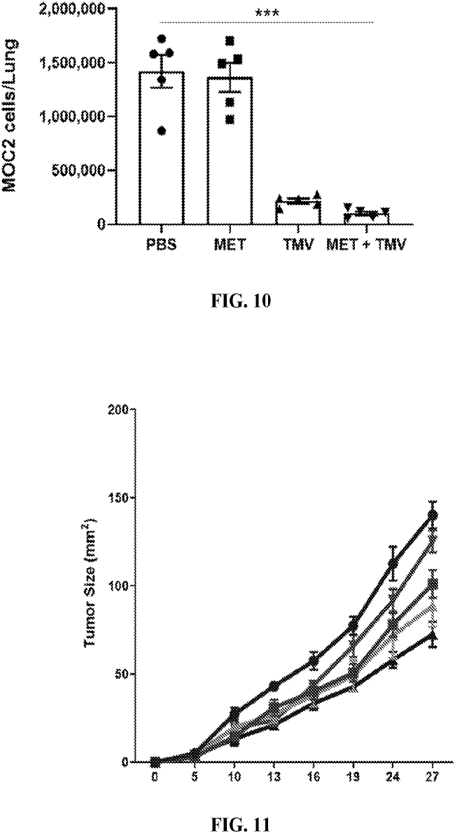

In the murine head and neck MOC2 cancer model, the TMV vaccine generated from MOC2 tumors was administered at Day 3, while MET was started at Day 3 and continued every other day until end point. The MOC2-challenged mice that were treated with MET alone had no reduction in tumor growth, while the TMV treated had a significant decrease in tumor area. Importantly, the combination therapy in this model yielded the best outcomes as evidenced in FIG. 9A and FIG. 9B. The lungs from these MOC2-challenged mice were harvested after 25 days of tumor growth and the number of metastatic cells was quantified after suspending the lungs into a single cell suspension (FIG. 10). Met alone did not have a significant reduction in lung metastatic cells, however both TMV alone and the combination of TMV and MET had a significant decrease in the number of metastatic MOC2 cells per lung.

### Example 2. TMV and Metformin Combination Therapy as a Post-Surgery Regimen to Treat Cancer

Mice were divided into four groups of five mice per group, wherein each group was to be treated with either a PBS control, metformin (MET) alone, 4T1-derived TMV (as described in Example 1) or TMV+MET. Each mouse was challenged with 4T1 mouse breast cancer cells by subcutaneous injection and tumors were later surgically resected (Day 0). On Day 5, mice in the MET and TMV+MET groups were administered 50 mg/kg metformin in drinking water daily until Day 20. On Days 5 and 12, mice in the TMV and TMV+MET groups were administered a dose of TMVs formed from 4T1 cells. Mice were then analyzed for survival (FIG. 8). Results showed that while MET alone and TMV alone increased mouse survival post-surgery, the TMV+MET combination therapy increased survival more extensively than either treatment alone.

### Example 3. TMV, Metformin, and Immune Checkpoint Inhibitor (ICI) Combination Therapy to Treat Cancer

Studies are conducted to determine the effect of metformin on TMV vaccination-induced antitumor immune responses in non-tumor bearing mice. Groups of mice are treated according to Table 1. TMV-treated mice are treated with 100 µg of 4T1 TMV vaccine prepared from 4T1 membrane vesicles from tumor tissue harvested from a different set of mice. TMVs are incorporated with GPI-mB7-1 and GPI-mIL-12 by protein transfer. Metformin is administered in the drinking water at a dose of 5 mg/ml (Eikawa, et al., PNAS, 112(6) (2015)). The immune checkpoint inhibitor (ICI) anti-PD-1 mAb are administered i.p. at 100 µg per dose after 1 and 4 days post-vaccination.

TABLE 1

| | | |
|---|---|---|
| | Treatment | Challenge |
| Group 1 | PBS | 4T1 |
| Group 2 | MET | 4T1 |
| Group 3 | ICI | 4T1 |
| Group 4 | TMV | 4T1 |
| Group 5 | TMV + ICI | 4T1 |
| Group 6 | TMV + MET | 4T1 |
| Group 7 | MET + ICI | 4T1 |
| Group 8 | TMV + ICI + MET | 4T1 |

Anti-tumor response elicited by TMV in the absence of tumor antigens shed by growing tumor cells, which may influence the dosing estimate, is analyzed. At day 3, 7 and 13 post-vaccination, the serum IgG response is assessed using capture ELISA, and the relevant cytokine response in peripheral blood is assayed using multiplex assays. At day 14 post-vaccination, mice are challenged with 20,000 4T1 cells subcutaneously into the mammary fat pad. Tumor size is measured twice per week. After 22 days, metastasis to the lungs is quantified using histological analyses of H&E stained sections or clonogenic assays using 6-TG containing medium since 4T1 cells are resistant to 6-TG. Simultaneously, the anti-tumor response of tumor infiltrating lymphocytes as well as peripheral lymphoid organs (spleen) is determined. Intracellular IFN-γ, TNF-α, IL-2, FOXP3 and Granzyme B levels are measured along with surface activation markers CD25, CD69, CD44, CD62L and CD107a on CD4 and CD8 T cell subsets using flow cytometry. In a separate experiment, survival is monitored for 3 months and tumor-free mice are re-challenged with 4T1 tumor cells to determine protective anti-tumor memory response.

In the 4T1 model where mice were challenged and treated with MET, TMV vaccine, and PD-1 blockade 3 days after implantation. Tumor growth inhibition was obtained with the TMV vaccine and PD-1 blockade, but combination treatment of metformin, TMV vaccine and PD-1 blockade resulted in the most effective control of primary tumor growth compared to monotherapies or PBS control (FIG. 11).

### Example 4. TMV, Metformin, and Immune Checkpoint Inhibitor (ICI) Combination Therapy as a Post-Surgery Regimen to Treat Cancer

Effects of metformin administration in a neoadjuvant therapeutic (pre-surgery) or an adjuvant therapeutic (post-surgery) setting using mice with established metastasis is examined. To investigate the effect of metformin on TMV vaccination, 20,000 4T1 cells are orthotopically implanted into the mammary fat pad of BALB/c mice. After 10 days (which is sufficient for establishment of lung metastasis in this model) the tumor is resected surgically, and mice are subjected to treatments outlined in Table 2. Two doses of 100 µg TMV vaccine are administered subcutaneously: one priming dose 2 days after surgery and a booster dose 9 days after surgery. The ICI (anti-CTLA-4 mAb) is administered i.p. at 100 µg per dose after 1 and 4 days post-TMV treatment. The survival is monitored following IACUC endpoint guidelines (weight loss and overall sickness due to metastasis). Metastasis is assessed using histological analyses or clonogenic assays.

TABLE 2

| | | |
|---|---|---|
| | Pre-surgery | Post-surgery |
| Group 1 | Water | PBS |
| Group 2 | Water | TMV |
| Group 3 | Water | ICI |
| Group 4 | Water | MET |
| Group 5 | Water | Metformin + ICI |
| Group 6 | Water | TMV + ICI |
| Group 7 | Water | TMV + MET |
| Group 8 | Water | TMV + ICI + MET |
| Group 9 | MET | PBS |
| Group 10 | MET | TMV |
| Group 11 | MET | ICI |
| Group 12 | MET | MET |
| Group 13 | MET | MET + ICI |
| Group 14 | MET | TMV + ICI |
| Group 15 | MET | TMV + MET |
| Group 16 | MET | TMV + ICI + MET |

To understand the effect of metformin on the systemic immune response elicited by TMV combination treatments, blood is analyzed 5 days after treatment in the survival experiment. Blood is analyzed using flow cytometry to identify immune cell population such as B cells, T cell subsets, NK cells, DCs, regulatory T cells, and myeloid derived suppressor cells. The activation of professional antigen presenting cells, such as DCs, is assessed using the activation markers MHC I, MHC II, CD80 and CD86. 4T1 specific cytokine response will be determined by ELISpot and intracellular cytokine staining of T cells isolated from tumor and spleen from a separate set of mice as described in Example 3. Furthermore, key activation markers such as CD69, CD44, CD62L KLRG-1 and CD107a are analyzed on peripheral as well as intra-tumor CD4 and CD8 T cells, while markers such as MHC I and PD-L1 are assessed on tumor cells using flow cytometry. Additionally, the serum IgG response is analyzed using flow cytometry for anti-tumor reactive antibodies.

To determine the direct effect of metformin on cancer cells, in vitro experiments are carried out using cultured 4T1 cells labelled with CFSE dye. Varying concentrations of metformin are added and assessed for proliferation using CFSE dilution measured by flow cytometry. Surface markers of interest such as surface MHC I and PD-L1 expression are quantified by flow cytometry.

Publications cited herein are hereby specifically incorporated by reference in their entireties and at least for the material for which they are cited.

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

1. A method for treating a subject having, or at risk of having, a cancer, comprising administering to the subject a therapeutically effective amount of a tumor membrane vesicle (TMV) and a metformin.

2. The method of claim 1, wherein the TMV comprises a lipid membrane, and a B7-1 and/or IL-12 molecule anchored to the lipid membrane.

3. The method of claim 1, wherein the TMV comprises the B7-1 and the IL-12 molecule anchored to the lipid membrane.

4. The method of claim 1, wherein the TMV comprises a lipid membrane and an antigen molecule anchored to the lipid membrane.

5. The method of claim 4, wherein the antigen molecule is selected from HER-2, PSA, and PAP.

6. The method of claim 4, wherein the antigen molecule is HER-2.

7. The method of claim 1, further comprising administering an immune checkpoint inhibitor.

8. The method of claim 1, further comprising administering one or more of an anti-CTLA4 antibody, an anti-PD1 antibody, and an anti-PD-L1 antibody.

9. The method of claim 8, wherein the anti-CTLA4 antibody is administered.

10. The method of claim 8, wherein the anti-PD1 antibody is administered.

11. The method of claim 8, wherein the anti-PD-L1 antibody is administered.

12. The method of claim 1 to 11, wherein the cancer is selected from the group consisting of lymphoma, B cell lymphoma, T cell lymphoma, mycosis fungoides, Hodgkin’s Disease, myeloid leukemia, bladder cancer, brain cancer, nervous system cancer, head and neck cancer, squamous cell carcinoma of head and neck, lung cancer such as small cell lung cancer and non-small cell lung cancer, neuroblastoma, glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers, testicular cancer, colon cancer, rectal cancer, prostatic cancer, and pancreatic cancer.

13. The method of claim 1, wherein the cancer is a breast cancer.

14. The method of claim 1, wherein the cancer is a triple negative breast cancer.

15. The method of claim 1, wherein the cancer is a squamous cell carcinoma.

16. The method of claim 1, wherein the cancer is lung cancer.

17. The method of claim 1, wherein the method reduces metastasis of the cancer.

18. The method of claim 1, wherein the method reduces the size of a tumor.

19. The method of claim 1, wherein the method reduces the amount of myeloid-derived suppressor cells in the subject’s blood or tumor.

20. The method of claim 1, wherein the method increases the amount of CD8+ T cells in a tumor.

21. The method of claim 1, wherein the method increases the amount of pro-inflammatory cytokine production in a tumor.

22. The method of claim 1, wherein the subject is a human.

23. The method of claim 1, wherein the method is performed after surgical resection of a tumor.

24. The method of claim 1, wherein the TMV is administered separately from the metformin.
